(19) United States
(12) Patent Application Publication
(10) Pub. No.: US 2024/0268847 A1
(43) Pub. Date: Aug. 15, 2024

(54) MEDICAL INSTRUMENT HAVING A SPRING UNIT OPTIMISED FOR CLEANING

(71) Applicant: Aesculap AG, Tuttlingen (DE)

(72) Inventors: Pedro Morales, Tuttlingen (DE); Dieter Weisshaupt, Immendingen (DE)

(21) Appl. No.: 18/563,645

(22) PCT Filed: May 23, 2022
(86) PCT No.: PCT/EP2022/063925

§ 371 Date: Nov. 22, 2023

(30) Foreign Application Priority Data
May 26, 2021 (DE) 10 2021 113 518.4

(51) Int. Cl. A61B 17/28

(52) U.S. Cl. CPC A61B 17/2841; A61B 2017/2845

(57) ABSTRACT

A medical instrument includes two handle elements that are pivotable relative to one another and a spring unit having two spring end portions. Each spring end portion is connected to one of the handle elements such that, when at least one of the handle elements is pivoted out of a starting position, the spring unit can pivot the handle element back into the starting position. The spring unit provides a substantially constant spring force when the handle elements are pivoted, and includes a first spring leg and a second spring leg. A platform-like projection is formed on at least one of the spring end portions and/or on at least one of the handle elements to connect the spring end portion to the associated handle element, such that the at least one spring end portion is raised relative to the associated handle element, with a gap formed between them.

### CROSS-REFERENCE TO RELATED APPLICATIONS

This application is the United States national stage entry of International Application No. PCT/EP2022/063925, filed on May 23, 2022, and claims priority to German Application No. 10 2021 113 518.4, filed on May 26, 2021. The contents of International Application No. PCT/EP2022/063925 and German Application No. 10 2021 113 518.4 are incorporated by reference herein in their entireties.

### FIELD

The present disclosure relates to a medical, in particular surgical, instrument, preferably a manually operable hand instrument, having at least two (a first and a second) gripping elements which are pivotably mounted relative to each other, and a spring unit/spring elastic assembly which has two spring end portions (at the spring ends), which are each connected to one of the two gripping elements, so that when at least one of the two gripping elements is pivoted out of a base position (against an elastic force of the spring unit), pivoting back into the base position can be carried out/realized/achieved via the spring unit.

### BACKGROUND

In medical or surgical (hand) instruments, return springs are currently used that are configured as leaf springs and lie in contact with a branch or a gripping element over a large area. Both the leaf spring and the gripping element have large, continuous surfaces facing each other which, starting from the common contact supporting surface, move continuously away from each other. It can also be said that the leaf spring and the gripping element have a semi-circular/arc-shaped contour with different radii in the portion of the fastening and successively increase the distance between them (from zero). While a partial surface of the leaf spring is in direct contact with a partial surface of the gripping element, the two surfaces move away from each other on the outside and form a very small gap between them, which gradually increases in size. As a result, the medical instruments are very difficult to clean and sterilize in the area of the contact supporting surface. In addition, the area where the return spring and gripping element lie against each other is exposed to contact corrosion or bimetal corrosion.

DE 10 2017 114 260 A1, for example, discloses a medical hand instrument in which a one-piece return spring is attached to a gripping element via form-fitting. However, the disadvantage of this instrument is that complex milled receiving portions have to be provided on the branch or gripping element. In addition, a one-piece return spring is provided so that a linearly increasing, high spring force is created when the hand instrument is closed. This spring force counteracts a force to be applied by a user, such as a surgeon, in particular in a closed position, and thus reduces the maximum force that can be applied manually. Furthermore, the spring is subject to a high degree of deformation, so that there is an increased risk of the return spring breaking during continuous use.

### SUMMARY

The objects and objectives of the present disclosure are to avoid or at least reduce the disadvantages of the prior art and in particular to provide a medical, in particular surgical, instrument which offers exceptionally good cleanability, is simple and inexpensive to manufacture and yet at the same time maintains a constant force for pivoting the gripping elements over a large, in particular an entire, travel range of the instrument. Furthermore, the instrument should in particular be small, safe, have a simple design, be easy to handle and simplify maintenance, in particular replacement of the spring unit.

The present disclosure thus relates to a medical instrument with two gripping elements (handle brackets, handle levers, lever arms) which are pivotable relative to each other, and a spring unit or spring-elastic assembly which is arranged between the gripping elements. The preferably U-shaped or V-shaped spring unit has two (spring) ends, the immediately adjacent area of which is referred to as the end portion. In particular, the spring end forms the spring end portion. Each of the two spring end portions is attached to a corresponding/associated one of the two gripping elements, so that when at least one of the two gripping elements pivots out of a base position, pivoting back into this base position is realized. This means that if one of the two gripping elements is pivoted manually by a user of the instrument, the spring unit can pivot the pivoted gripping element back to its base position as soon as the user releases the pivoted handle part. Preferably, the instrument has load arms opposite a hinge that pivotably connects the gripping elements. The medical instrument may also be configured in such a way that both gripping elements are pivoted when the instrument is used. If only one gripping element is intended to be pivoted, the other gripping element only serves as a counter support for the user's hand during pivoting. In particular, the medical instrument has a distal effective portion which is actuatable via the proximal gripping portion, such as a clamping portion (clamping/gripping branches) or cutting portion (cutting blades), etc.

According to the present disclosure, the instrument is thus provided with such a spring unit, which is adapted to provide an approximately constant force/spring force/restoring force when the gripping elements are pivoted, in particular over an entire closing travel of the instrument. Preferably, an at least two-part spring unit is provided in the instrument for realization, which has at least a first spring portion, in particular a first spring leg, and a (separate) second spring portion, in particular a second spring leg, which are connected to each other, in particular at least partially movably coupled to each other. When the two legs or gripping elements are pressed together from the base position, a spring force of the spring unit (when pivoting into a closed position) can be maintained uniformly and homogeneously, in particular over the entire travel range of the closing action. There is no linear increase in the spring force. This special design supports the user of the instrument and ultimately also increases the potentially maximum closing force that can be applied and counteracts user fatigue.

In addition, the spring unit is connected to the gripping elements in a specially adapted manner. Specifically, at least one part of the spring unit is connected to at least one spring end portion, in particular the spring end, on or via a platform-like/platform-shaped projection or a platform on the associated gripping element of the instrument, in particular attached. This creates a defined (minimum) distance between the portion of the spring, in particular an entire spring leg, and the associated gripping element in the area of the platform-shaped projection and thus of the spring end portion. The gap formed in this way ensures a minimum clearance between the spring leg and the gripping element, at least in the base position, so that good cleaning of the instrument is guaranteed. The platform-shaped projection may also be used to define a geometric transition between the spring unit and the gripping element, for example by rounding off edges in order to further improve cleanability. Manufacturing the instrument is significantly simplified.

In other words, at least one spring portion, in particular the spring leg, of the spring unit is rigidly attached to the associated/corresponding gripping element via an intermediate/interposed platform-shaped projection/step/socket/spacer/bench, which in particular forms a (single) defined contact surface via which the spring portion, in particular the spring leg, and the associated gripping element are in contact with each other in at least the base position, so that they have a gap of a minimum size or a predefined distance between them. In particular, the spring unit is only connected to the gripping elements via the platform-shaped projection(s) and is in contact with them. The cleanability and manufacture of the instrument is improved.

In yet other words, the spring unit is adapted to provide an essentially constant spring force when the gripping elements are pivoted. In particular, the spring unit is configured in at least two parts and has a first part, in particular a first spring leg, and a second part, in particular a second spring leg, in order to ensure a uniform spring force. Furthermore, a platform-shaped projection is formed/shaped/molded on at least one of the two spring end portions and/or on at least one of the two gripping elements, via which the spring end portion is rigidly connected to the associated gripping element, so that the at least one spring end portion is arranged raised/offset relative to the associated gripping element in at least the base position and forms a gap between them or has a distance from the gripping element caused by the platform-shaped projection.

In yet other words, the medical instrument, preferably of the forceps or punch type, has at least two branch/instrument parts/legs which are pivotably mounted relative to each other and have a proximal gripping portion/actuating portion as viewed in the longitudinal direction of the instrument. The instrument has a spring-elastic return unit/spring unit, in particular in the form of a return spring, which is provided and/or molded between the first branch and the second branch, preferably in the region of the gripping portion, in such a way that it holds the first branch and the second branch in a base position or pre-tensions them into this position. A platform-shaped projection/step/socket is provided between at least the first branch and a first spring portion of the spring unit, via which they are connected to each other and which is formed on the first branch and/or on the first spring portion. Alternatively or additionally, between the second branch and the second spring portion, there may also be a platform-shaped projection/step/socket via which they are connected to each other and which is formed on the second branch and/or on the second spring portion. The intermediate spaces can be cleaned very easily.

The term base position defines a position of the pivotable gripping elements in relation to each other, into which they move without external (manual) force, only due to the spring unit. In particular, this base position may be a (geometrically determined) maximum opening position of the instrument.

The term platform-shaped projection defines a geometry similar to a platform that is suitable for raising/elevating/lowering/displacing a second surface (raised surface) relative to a first surface (base surface, surrounding surface, basic surface) in a direction essentially orthogonal to the first surface at this point. It can also be said that the platform-shaped projection achieves a gradation (with the individual form of a step, in particular a block step).

The term spring end portion defines a portion of the spring end. In particular, the spring end may be the spring end portion.

Advantageous embodiments are explained below.

According to one aspect of the present disclosure, a spring unit may be formed in two parts in the form of two separate leaf springs or two spring steel wires, which are distally connected to each other, in particular via a distal fork-nose connection or a distal sphere-pan connection. The first leaf spring forms the first spring leg and the second leaf spring forms the second spring leg. The two-part design keeps the number of components small and minimizes gap formation in contact areas. The leaf springs are inexpensive and easy to manufacture and to assemble. In particular, in a top view of the instrument or in a side view of the leaf springs, the two leaf springs have a convex arc shape which tapers to a point/arrow shape at the distal connection point.

According to a further aspect of the disclosure, the platform-shaped projection may form a front-face contact supporting surface to which or via which the spring end portion or the spring unit is attached to the associated gripping element. If the spring end portion or the spring unit and the associated gripping element are separate components, the contact supporting surface creates a defined interface or connection point on which the spring end portion or a surface of the gripping element rests. The term front face means that the surface is formed on the raised side (i.e. the surface at a distance from the base surface). The base surface may be the relevant surface of the spring end portion if the platform-shaped projection is formed on the spring end portion, and/or may be the relevant surface (around the platform-shaped projection) of the gripping element if the platform-shaped projection is formed on the gripping element. The contact supporting surface itself may assume different surface shapes, i.e. it may also be slightly curved (convexly or concavely) or curved or wavy.

Preferably, the platform-shaped projection may be block-shaped with a block-base surface that extends perpendicular to the relevant base surface in the direction of a height of the platform-shaped projection and forms the contact supporting surface on the front face. The height of the platform-shaped projection is in particular perpendicular to a longitudinal axis of the gripping element (at this point) or to a longitudinal axis of the spring end portion (at this point). In particular, the block-base surface may have an essentially rectangular basic shape. Alternatively, the basic shape may also have an oval basic shape. Preferably, the outer contour of the block-base surface is convex, so that there are no undercuts on side surfaces. The side surfaces extend around the contact supporting surface or the block-base surface at least in the direction of the height of the platform-shaped projection, thus forming the side surfaces of the platform-shaped projection.

According to a further aspect of the disclosure, at least one of the two spring end portions may be connected to the gripping element via the platform-shaped projection via a form-fitting connection, in particular a screw connection and/or a firmly bonded connection, in particular via gluing and/or welding and/or soldering. In the case of a screw connection, a cost-effective assembly is realized which connects the spring unit securely and durably to the gripping elements and the spring unit can be easily dismantled and replaced. In the case of gluing, welding or soldering, gaps that inevitably occur in the area of the connection can be hermetically sealed and a closed surface can be ensured. This supports cleaning and sterilization.

In the case of the screw connection, the screw in particular may be screwed into a complementary blind hole/blind hole bore with an inner thread from the spring end portion in the direction of the associated gripping element, so that the gripping element does not have a drilled hole on an outer gripping surface. In other words, a screw is screwed into the gripping element, in particular perpendicular to and through the spring end portion. If the platform-shaped projection is formed on the gripping element, the screw can be screwed into an inner thread of the projection provided in a blind hole. This avoids a sharp edge on the outside of the gripping surface/engagement surface, which represents a dangerous area for tearing open a surgical glove. Likewise, a flat surface of the outer gripping surface is not interrupted and the surface is kept closed. Alternatively or additionally, the screw may preferably also be screwed in from the side of the gripping element in the direction of the spring end portion. In particular, the platform-shaped projection may be formed on the spring end portion and the screw can be screwed into the projection so that it does not protrude through the spring end portion and an averted surface of the spring end portion is not interrupted or closed. This alternative screw connection simplifies both assembly and replacement of the spring unit.

According to a further embodiment, the platform-shaped projection may have a height of at least 1 mm, preferably at least 2 mm, particularly preferably at least 4 mm. The height defines a distance between a base point (on the base surface) of the gripping element and, to a certain extent, a base point of the spring end portion of the platform-shaped projection. This distance is measured in particular perpendicular to a longitudinal instrument axis of the gripping element at the location of the platform-shaped projection. In particular, the height is the distance on the distal side of the platform-shaped projection, i.e. the side facing the bearing. In particular, the height is a distance between the front-face contact supporting surface of the platform-shaped projection and its base or a base surface/basic surface of the gripping element facing the spring unit, so that a gap forming between the gripping element and the spring end portion in at least the base position of the instrument also has at least this height. This minimum clearance dimension or height ensures sufficiently good cleanability.

In particular, the platform-shaped projection may have a maximum height of 5 mm, so that during a closing movement (from the base position) the two spring end portions do not contact each other even in a (maximum) closed position.

Preferably, a contact supporting surface of the platform-shaped projection may be at least 5 mm² (mm\hat{ }2/square millimeter) and/or at most 1 cm² (cm\hat{ }2/square centimeter) and/or may be flat. The minimum surface dimension ensures sufficient stability of the connection between the gripping element and the spring end portion. The maximum surface dimension is used to ensure that a construction dimension remains compact and sufficient spring force remains.

According to one aspect of the disclosure, an angle between a side of the spring end portion facing the associated gripping portion, which in particular rests on the contact supporting surface, and a side surface of the platform-shaped projection may be at least 20°, preferably at least 40°, particularly preferably more than 65° and very particularly preferably 90°. With this minimum angle, a gap is sufficiently blunt and not too tapered. In particular, an angle between the base surface/(basic) surface of the gripping element facing the spring unit and a side surface of the platform-shaped projection may alternatively or additionally be at most 140°, preferably at most 110°, particularly preferably 90°. A portion of the side wall may also have an arcuate shape/chamfer extending towards the gripping element and/or the spring end portion, in particular towards the front-face contact surface, in order to avoid an edge and increase cleanability. In other words, at least one edge of the platform-shaped projection touching the gripping element or the spring end portion may be chamfered (a haunch).

According to a further aspect of the present disclosure, the platform-shaped projection may comprise an alignment element, in particular in the form of a protruding stop or a pin, in order to align the spring end portion with respect to the gripping element, if formed separately and resting on the contact supporting surface.

In particular, the facing outer contours and/or surfaces of at least one of the two gripping elements and the associated spring leg run essentially parallel to each other in the area of the platform-shaped projection. In particular, they run parallel to each other distal to the platform-shaped projections. In other words, the longitudinal handle axis and the longitudinal axis of the spring leg run essentially parallel to each other in the area distal to the platform-shaped projections, in particular up to a length of 30%, preferably up to a length of 50% of the total length of the spring leg. In particular, if a leaf spring is used as a spring leg, the leaf spring and gripping element have an approximately identical or parallel course in at least one position.

In a further embodiment, the platform-shaped projection may have a length (in the direction of the longitudinal axis of the gripping element or in the direction of the longitudinal axis of the spring end portion) of at least 5 mm, preferably at least 10 mm. This minimum length achieves sufficient mechanical strength or contact supporting surface to prevent bending of the spring unit or detachment from the gripping element.

In particular, the platform-shaped projection has a side surface, especially a distal side surface, which is perpendicular to the contact supporting surface.

In one embodiment, at least one spring leg, the platform-shaped projection and the associated gripping element may be formed integrally with each other.

Preferably, the at least one spring end portion is connected to the associated gripping element such that the spring unit deforms only in the pivot plane.

In other words, the present disclosure preferably relates to a medical instrument which is characterized in that a spring device/spring unit with in particular two leaf springs, one in each branch, are mounted in an elevated position on a bench (platform-shaped projection), preferably screwed together. Since the spring unit, in particular the two leaf springs, are kept at a distance (from the associated gripping element) by the benches (platform-shaped projections), good cleanability, such as sterilizability, can be achieved. In particular, the spaces in between can be cleaned very well or without any problems. The contact surface (of one gripping element with the spring element) is limited to the length of the bench (platform-shaped projection). If in particular a two-part spring is used for the medical instrument, this has the advantage that the spring force does not increase linearly when the instrument is closed, in particular with forceps. As a result, the closing forces remain virtually constant. In particular, the two-part spring is in contact distally via a spring force, preferably through a fork/nose connection or a ball/socket connection.

### BRIEF DESCRIPTION OF THE DRAWINGS

The present disclosure is explained in more detail below by reference to preferred embodiments with reference to the accompanying Figures. The following is shown:

FIG. 1 shows a plan view of a distal gripping portion of a medical instrument of a first preferred embodiment, in which a spring unit rests on and is attached to platform-shaped projections, each of which is formed on a leg;

FIG. 2 shows a plan view of a medical instrument of a second preferred embodiment, in which platform-shaped projections are formed on the spring unit itself, which rest on and are attached to the gripping elements;

FIG. 3 shows a plan view of a medical instrument of a third preferred embodiment, which is a combination of the two embodiments of FIGS. 1 and 2;

FIG. 4 shows a detailed partial view of a medical instrument of a further, fourth preferred embodiment, in which the platform-shaped projection is formed on the spring unit and is fastened with a screw connection from the outside on the side of the gripping element;

FIG. 5 shows a plan view of a medical instrument of a further, fifth preferred embodiment, in which the platform-shaped projections are formed on the spring unit which rest on the gripping elements and are welded on; and

FIG. 6 shows a detailed partial view of a connection point of a medical instrument of a further, sixth preferred embodiment, in which the connection between the spring unit and the platform-shaped projections of the gripping element is realized firmly bonded via an adhesive connection.

The Figures are schematic in nature and are only intended to aid understanding of the present disclosure. Identical elements are provided with the same reference signs. The features of the various embodiments can be interchanged.

### DETAILED DESCRIPTION

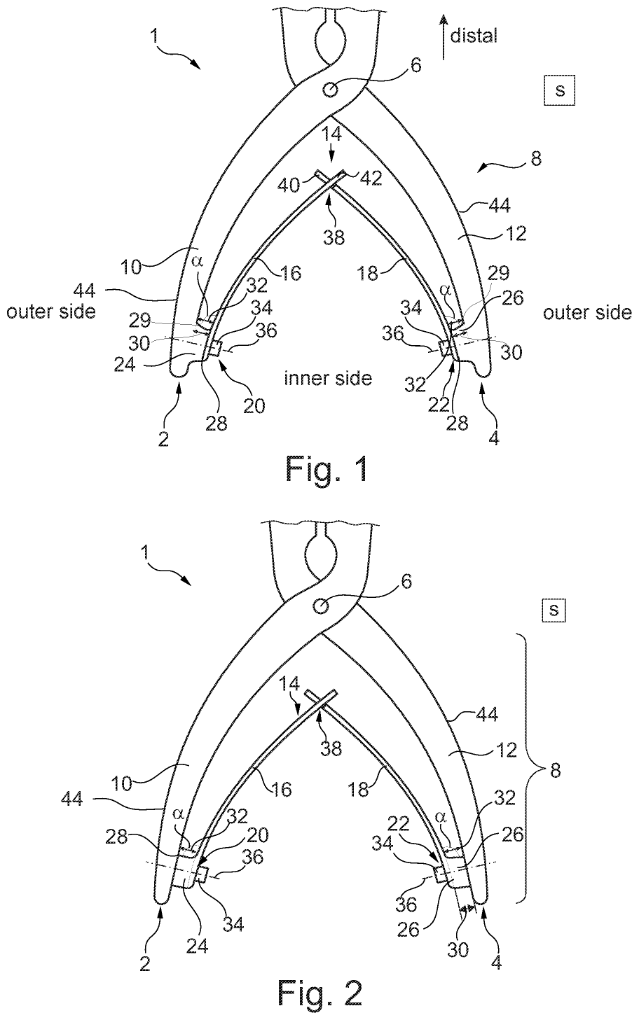

FIG. 1 shows a partial view of a medical instrument 1 of a first preferred embodiment. The instrument 1, in the form of a hand instrument, is of a forceps design. For this purpose, the instrument 1 has two levers 2, 4, which are pivotably connected to each other via a hinge 6. This allows the two levers 2, 4 to be pivoted in a pivot plane S in relation to each other, similar to a pair of scissors or grasping forceps. The proximal portions of the levers 2, 4 (proximal to the hinge 6 and pointing towards the user) form a gripping portion 8 with corresponding gripping elements or handles 10, 12, which are essentially symmetrical to each other with respect to a longitudinal axis of the instrument (proximal-distal). The gripping portion 8 is also symmetrical to the pivot plane S.

A two-part spring unit 14 in the form of a two-part leaf spring is provided between the first handle 10 and the second handle 12, which is essentially V-shaped with a slight bulge, tapers distally in the shape of an arrow and has two spring legs 16, 18, which form the leaf springs. When at least one of the two handles 10, 12 is pivoted from a base position of the instrument against an elastic force of the spring unit 14, pivoting back into the base position is realized via the spring unit 14. At their respective free spring end portions 20, 22, the spring legs 16, 18 rest on platform-shaped/platform-like projections/steps 24, 26 that face each other and are formed on inner sides/inner surfaces of the handles 10, 12 that face each other. The first platform-shaped projection 24 and the first handle 10 as well as the second platform-shaped projection 26 and the second handle 12 are formed or configured integrally/in one piece. The platform-shaped projection 24, 26 raises the two spring end portions 20, 22 in a defined manner relative to the surroundings, in this case the inner sides or inner surfaces (base surfaces) of the handles 10, 12, and a minimum distance is formed between the respective handle 10, 12 and the respective spring leg 16, 18.

Due to the special two-part design of the spring unit 14 as a return spring, a substantially constant spring force can be provided, on the one hand, for good handling and durability when the two levers 4, 6 or handles 10, 12 are pivoted in the pivot plane S over the entire closing path and, on the other hand, a simple design and cost-effective manufacture. The special connection of the first and second spring leg 16, 18 via the platform-shaped projection 24, 26 ensures good cleanability as well as a simple design and cost-effective manufacture of the instrument 1.

Specifically, the two leaf springs of the spring unit 14 each lie flat on a front-face contact supporting surface 28 of the platform-shaped projection 24, 26. The contact supporting surface 28 defines the only structural connection between the spring unit 14 and the respective handles 10, 12. A height 30 of the platform-shaped projections 24, 26, which is essentially perpendicular to a longitudinal handle axis, can be used to set the dimension of a gap or a gap width 32 that forms between the spring leg 16, 18 and the handle 10, 12. The gap width 32 is measured from the inner surface of the handles 10, 12 (as the base surface) to the opposite surface of the spring leg 16, 18 (raised surface; corresponds to the contact supporting surface 28). In particular, one height is 3 mm and therefore the smallest gap width 32 shown in FIG. 1 is also 3 mm.

In order to fix the leaf springs firmly on the platform-shaped projections 24, 26, a screw 34 with a longitudinal screw axis 36 is screwed on the inside from the side of the leaf springs in the direction of the height 30 of the projection 24, 26 or essentially perpendicular to a longitudinal handle axis or perpendicular to the contact supporting surface 28 outwards into a corresponding inner thread of a blind hole (not shown) in the handle 10, 12. This enables simple assembly and, if necessary, also disassembly, for example for maintenance or replacement of the spring unit 14. In addition, the instrument 1 is also very easy to manufacture and can be assembled without errors and without a great deal of prior knowledge.

The two-part spring unit 14 connects or couples the two spring legs 16, 18 to each other via a distal spring coupling 38. In this first embodiment, the spring coupling is configured in the form of a fork-nose connection, in which the first spring leg 16 has a nose 40 and the second spring leg has a fork 42. The nose 40 protrudes into the fork 42 and is (slightly) displaceably and pivotably held by the fork 42. This allows the distal ends of the spring to pivot towards each other during pivot movement of the handles 10, 12 and ensures an essentially constant or uniform spring force along the entire closing path. Alternatively, a sphere-pan connection may of course also be used instead of the fork-nose connection. The spring unit 14 has an arrowhead shape with a tapered distal spring coupling 38 and bulging spring legs 16, 18.

An angle or gap angle α between an underside of the leaf springs (which faces the handles 10, 12) and a directly adjoining side surface 29 of the platform-shaped projections is 70° in this embodiment. In this way, a sufficiently obtuse angle is provided. In addition, a slight rounding towards the distal direction and on the sides of the contact supporting surface 28 is provided in order to support cleaning.

Since the screws 34 are screwed into a blind hole with an inner thread from the inside to the outside of the handles 10, 12 along the longitudinal screw axis 36, an external or outer gripping surface 44 has a flat and closed surface. This prevents contamination and damage to a surgical glove, since the glove has a sharp edge to prevent tearing.

The medical instrument 1 of a second preferred embodiment shown in FIG. 2 differs from the first preferred embodiment in that platform-shaped projections 24, 26 are not formed in one piece on the gripping elements 10, 12, but on the two spring legs 16, 18 of the spring unit 14. The spring legs 16, 18 are attached by their platform-shaped projections 24, 26, or more precisely by the contact supporting surfaces 28, to the inner surfaces of the handles 10, 12. In this embodiment, the spring leg 16, 18 are also screwed into the handles 10, 12 from the sides of the spring leg 16, 18 via screws 34, so that an outer gripping surface 44 has a closed surface without openings. In this way, standardized instruments or gripping elements can be used into which only a blind hole bore with inner thread is inserted. Only the spring unit 14 needs to be adapted accordingly to the inner surfaces of the handles 10, 12.

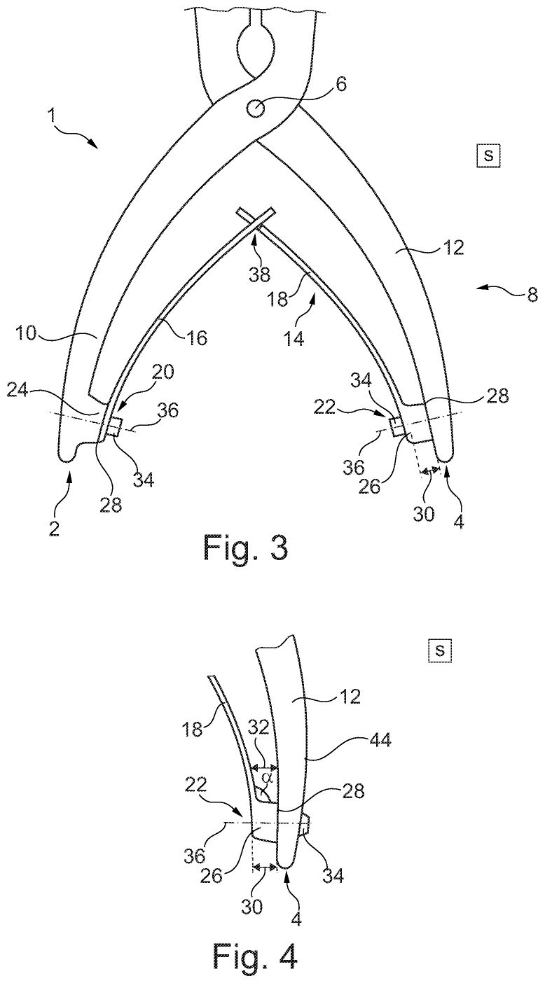

FIG. 3 shows a further, third embodiment of the instrument 1, in which a combination of the platform-shaped projections 28 of the first handle 10 of the first embodiment and the second handle 12 of the second embodiment is used. Specifically, a platform-shaped projection 24 is formed in one piece on the first handle 10, on whose contact supporting surface 28 the leaf spring or the first spring leg 16 rests and is attached, whereas the second handle 12 has no platform-shaped projection, but the second spring leg 18 has the second platform-shaped projection 26, which is formed in one piece on or with it.

In contrast to the first three embodiments, in FIG. 4 in a medical instrument 1 of a further preferred embodiment, the spring unit 14 is connected to the second handle 12 via a screw 34, which is screwed from the side of the outer gripping surface 44 of the second gripping element 12 (only one side of the instrument 1 is shown here for illustration purposes) in the direction of the spring end portion 22 into a blind hole bore with an inner thread formed in the platform-shaped projection 26. This design has the advantage that the screws can be easily screwed in from the outside and that a closed surface without an opening is provided on the inside of the spring leg or the spring end portion to prevent the formation of germs.

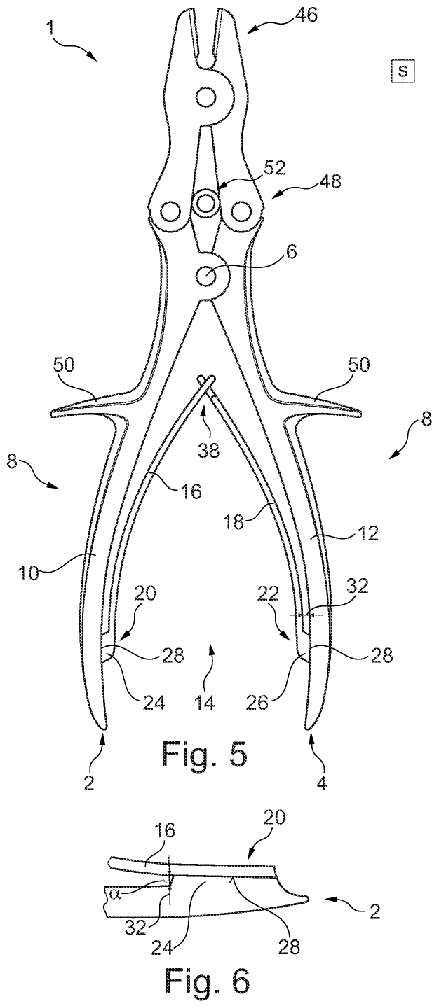

FIG. 5 shows a medical instrument 1 of a further preferred embodiment in the form of branch forceps with a plurality of hinges or a multi-link lever mechanism 48 and a distal forceps cutting portion 46. In contrast to the previous embodiments, the two platform-shaped projections 24, 26 formed on the spring unit 14 are not screwed on, but are firmly bonded on both sides to the handles 10, 12. In the present embodiment, these are welded to the handles 10, 12 in order to create a tight and long-lasting connection between the two components. In this embodiment, the height 30 of the platform-shaped projections 24, 26 and thus also the gap width 32 is reduced, as a minimum screw-in depth does not have to be guaranteed. Nevertheless, the platform-shaped projection 24, 26 ensures a minimum clearance in the cleaning position shown in FIG. 5, so that the instrument 1 can be cleaned easily. In this embodiment, the cleaning position differs from the base position in that an insertable and removable sleeve 52 is provided in the lever mechanism 48, which opens the instrument 1 slightly so that the distal forceps portion 46 can also be cleaned easily. In the cleaning position of the instrument 1, the spring legs 16, 18 run in a similar shape to the handles 10, 12, but are slightly compressed proximally, and lie or run parallel to each other over a length of ⅓ of the total length, particularly in areas of the spring end portions 20, 22.

In order to further improve the ergonomics and manageability of the instrument, hand stops 50 are provided on the outer gripping surfaces 44, which project laterally outwards on the handles 10, 12 perpendicular to the longitudinal axis of the instrument and geometrically limit and protect a user's hand distally when gripping the instrument. They also provide a stop for applying force in the distal direction, for example when the instrument 1 has to be moved distally against resistance.

FIG. 6 shows a detailed view of a further preferred embodiment of an attachment/connection area of the spring end portion 16 on the plane contact supporting surface 28 of the handle 10 (only the left side of the instrument 1 is shown here as an example). The spring end portion 16 is bonded via an adhesive that meets medical technology requirements and also withstands sterilization.

The first platform-shaped projection 24 has a plane side surface 29 distally and a curved side surface 29 proximally. The gap angle α is 80° and an angle on the foot side of the platform-shaped projection is correspondingly 100°. A length of the contact supporting surface 28 seen in the direction of the longitudinal handle axis is 10 mm. This (minimum) length ensures a sufficient contact surface for the transmission of the spring forces and also provides a sufficient adhesive surface. In particular, the size of the contact supporting surface 28 is at least or approximately 50 mm².

The attachment between the handle 10 and the spring leg 16 shown in FIG. 6 can also be easily transferred to the embodiment shown in FIG. 5.

In a further preferred embodiment (not shown), a pin projecting out of and perpendicular to the contact supporting surface may additionally be provided on the platform-shaped projection, which protrudes into a complementary bore, similar to a pin-bore connection, and aligns the spring leg with respect to the handle, so that unintentional rotation of the spring unit is prevented.

1. A medical instrument comprising:
a first gripping element;
a second gripping element; and
a spring unit,
the first gripping element and the second gripping element being pivotably mounted relative to each other,
the spring unit comprising a first spring end portion and a second spring end portion, the first spring end portion being connected to the first gripping element, and the second spring end portion being connected to the second gripping element, so that when at least one of the first gripping element and the second gripping element is pivoted out of a base position, pivoting back into the base position can be carried out via the spring unit,
the spring unit being adapted to provide a substantially constant spring force when the first gripping element and the second gripping element are pivoted, and
a platform-shaped projection being formed on at least one of the first spring end portion, the second spring end portion, the first gripping element and the second gripping element, such that the platform-shaped projection at least one of:
rigidly connects the first spring end portion to the first gripping element, so that the first spring end portion is raised relative to and distanced from the first gripping element in at least the base position; and
rigidly connects the second spring end portion to the second gripping element, so that the second spring end portion is raised relative to and distanced from the second gripping element in at least the base position.

2. The medical instrument according to claim 1, wherein the spring unit is formed in two parts and comprises two leaf springs or two spring steel wires, which are coupled to each other via a distal fork-nose connection or a distal sphere-pan connection.

3. The medical instrument according to claim 1, wherein the platform-shaped projection has a height of at least 1 mm.

4. The medical instrument according to claim 1, wherein the platform-shaped projection forms a front-face contact supporting surface via which at least one of the first spring end portion and the second spring end portion is attached to the first gripping element and the second gripping element, respectively.

5. The medical instrument according to claim 4, wherein the platform-shaped projection is block-shaped.

6. The medical instrument according to claim 4, wherein the front-face contact supporting surface is at least 5 mm² and/or at most 1 cm² and/or is planar.

7. The medical instrument according to claim 1, wherein a gap angle between a side of the first spring end portion facing the first gripping element and a side surface of the platform-shaped projection is at least 20°.

8. The medical instrument according to claim 1, wherein at least one of the first spring end portion and the second spring end portion is connected to the first gripping element and the second gripping element, respectively, via the platform-shaped projection via a screw connection and/or by bonding.

9. The medical instrument according to claim 8, wherein the at least one of the first spring end portion and the second spring end portion is connected to the first gripping element and the second gripping element, respectively, via the platform-shaped projection via a screw connection, in which a screw is screwed into a blind hole bore with an inner thread from a side of said at least one of the first spring end portion and the second spring end portion in a direction of the first gripping element and the second gripping element, respectively, so that the first gripping element and the second gripping element does not have a drilled hole on an outer gripping surface, or that the screw is screwed in from a side of the outer gripping surface of the first gripping element and the second gripping element in a direction of the first spring end portion and the second spring end portion.

10. The medical instrument according to claim 1, wherein the platform-shaped projection comprises an alignment element configured to align the first spring end portion and the second spring end portion with respect to the first gripping element and the second gripping element, respectively.
